(19) United States
(12) Patent Application Publication
(10) Pub. No.: US 2007/0011828 A1
(43) Pub. Date: Jan. 18, 2007

(54) Novel double para-phenylenediamines joined by a branched aliphatic group and method of dyeing keratin fibers

(76) Inventors: Stephane Sabelle, Paris (FR); Eric Metais, St. Leu la Forêt (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/476,823

(22) Filed: Jun. 29, 2006

Related U.S. Application Data
(60) Provisional application No. 60/698,929, filed on Jul. 14, 2005.

(30) Foreign Application Priority Data
Jun. 29, 2005 (FR) 0551807

(51) Int. Cl. A61K 8/00

(52) U.S. Cl. 8/405

(57) ABSTRACT

Disclosed herein is a novel family of double para-phenylenediamines joined by a branched aliphatic linkage. Also disclosed herein is a dyeing composition comprising, in a medium suitable for dyeing, at least one para-phenylenediamine joined by a branched aliphatic linkage. Further disclosed herein is a method for dyeing keratin fibers comprising applying a composition of the present disclosure to the keratin fibers.

This application claims benefit of U.S. Provisional Application No. 60/698,929, filed Jul. 14, 2005, the contents of which are incorporated herein by reference. This application also claims benefit of priority under 35 U.S.C. § 119 to French Patent Application No. FR 05 51807, filed Jun. 29, 2005, the contents of which are also incorporated herein by reference.

Disclosed herein is a novel family of double para-phenylenediamines joined by a branched aliphatic linkage. Also disclosed herein are compositions for dyeing keratin fibers, for example, human keratin fibers such as the hair, comprising at least one double para-phenylenediamine joined by a branched aliphatic linkage. Further disclosed herein is a method of dyeing keratin fibers comprising applying a composition as disclosed herein to the keratin fibers.

It is common practice to dye keratin fibers, such as human hair, with dyeing compositions comprising oxidation dye precursors, generally called oxidation bases, such as ortho- or para-phenylenediamines, ortho- or para-aminophenols, and heterocyclic compounds. These oxidation bases are colorless or weakly colored compounds, which, when combined with oxidizing products can give rise, by a process of oxidative condensation, to colored compounds.

It is also known that it is possible to vary the shades obtained with these oxidation bases by combining them with couplers or dyeing modifiers, the latter being chosen, for example, from the aromatic meta-diaminobenzenes, meta-aminophenols, meta-diphenols, and certain heterocyclic compounds, such as indole compounds and pyridine compounds.

The great variety of the molecules available as oxidation bases and couplers may allow for a rich palette of colors to be obtained.

The so-called “permanent” dyeing obtained using these oxidation dyes ideally satisfies at least one of a number of characteristics. For example, the dye ideally does not present any problems of a toxicological nature, makes it possible to obtain the color shades of the desired intensity, has good resistance to external agents, such as light, weather, washing, permanent waving, sweat, and/or rubbing, provides coverage of white hair, displays minimal selectivity, i.e., ensures that the smallest possible differences in coloration are obtained along the same keratin fiber, which generally is differently sensitized (i.e., damaged) between its tip and its root.

The inventors have discovered, suprisingly, novel oxidation bases which may be capable of dyeing keratin fibers in a variety of strong, aesthetic shades with low selectivity, and which may be resistant to the various aggressive factors to which the fibers may be subjected, such as light, sweat, and/or shampoos.

Thus, disclosed herein is a novel family of double para-phenylenediamines of formula (I) and the corresponding addition salts thereof: 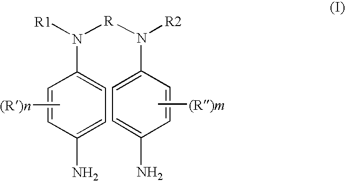
wherein:
R is chosen from branched C₃-C₁₅ alkylene radicals,
R₁ and R₂, which may be identical or different, are chosen from:
   hydrogen,
   linear and branched C₁-C₆ alkyl radicals, and
   linear and branched C₁-C₆ alkyl radicals substituted by at least one radical chosen from hydroxy, C₁-C₄ alkoxy, amino, C₁-C₄ monoalkylamino, and C₁-C₄ dialkylamino radicals,
R′ and R″, which may be identical or different, are chosen from:
   C₁-C₆ alkyl radicals,
   C₁-C₆ alkoxy radicals,
   C₁-C₆ hydroxy-alkoxy radicals,
   C₁-C₆ alkoxy(C₁-C₆)alkyl radicals, and
   C₁-C₆ mono- or poly-hydroxy alkyl radicals, and
n and m, which may be identical or different, are chosen from integers ranging from 0 to 4.

The para-phenylenediamines of formula (I) can make it possible to obtain very strong coloration of keratin fibers, have low selectivity, and are resistant to external factors, for example, light.

Also disclosed herein are compositions for dyeing keratin fibers, for example, human keratin fibers such as the hair, comprising at least one para-phenylenediamine of formula (I).

Further disclosed herein is a method for dyeing keratin fibers, for example, human keratin fibers such as the hair, comprising applying a composition of the present disclosure to the keratin fibers. Still further disclosed herein is a multi-compartment device or a dyeing “kit” comprising at least one first compartment comprising at least one composition of the present disclosure and at least one second compartment comprising at least one oxidizing composition.

Compounds

Para-phenylenediamine Compounds of Formula (I)

Non-limiting examples of para-phenylenediamines of formula (I) include, for example:

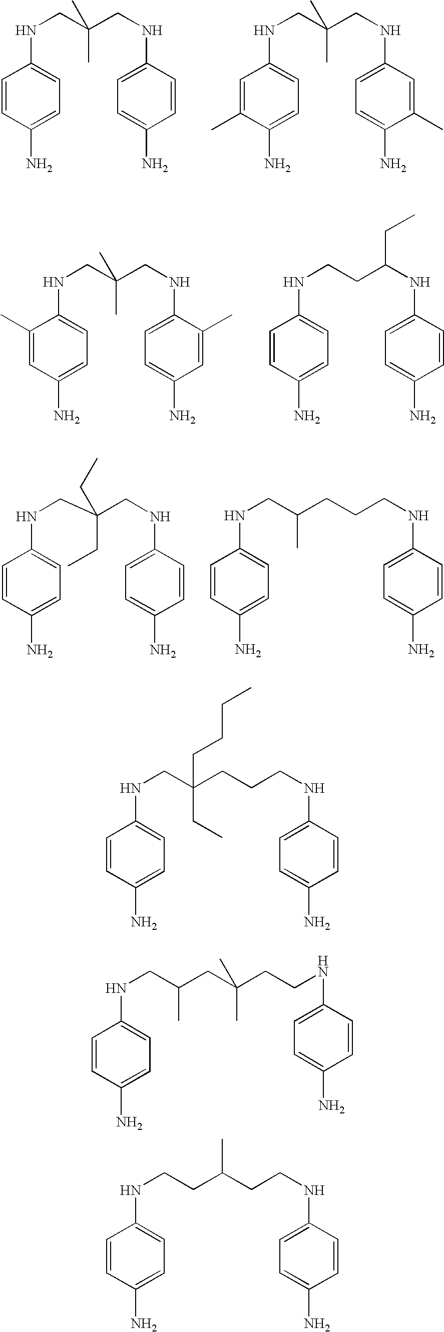

According to one embodiment of the present disclosure, in formula (I), R can be chosen from branched C₄-C₁₁ alkylene radicals. In another embodiment, R₁ and R₂, which may be identical or different, can be chosen from hydrogen and optionally substituted C₁-C₄ alkyl groups. In yet another embodiment, n and m, which may be identical or different, can be equal to 0 or 1.

Examples of suitable addition salts include, but are not limited to, acid addition salts, such as hydrochloric acid, hydrobromic acid, sulphuric acid, citric acid, succinic acid, tartaric acid, lactic acid, para-toluene-sulphonic acid, benzene-sulphonic acid, phosphoric acid, and acetic acid addition salts.

They may also be in the form of solvates, for example, hydrates and solvates of linear or branched alcohols, such as ethanol and isopropanol.

The para-phenylenediamines of formula (I) according to the present disclosure can be prepared according to conventional methods of synthesis, for example, those disclosed in German Patent Application No. DE 101 44 226 A.

For example, the para-phenylenediamines of formula (I) may be synthesized according to the following reaction scheme:

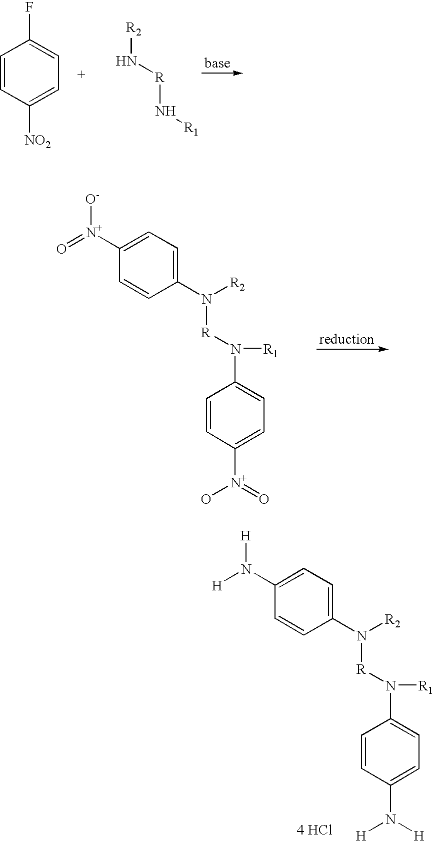

The first stage in the synthesis is a nucleophilic substitution of a diamine on a derivative of para-fluoro-nitrobenzene, a stage suggested, for example, by the publications Synthesis 12: 1147-1148 (1990) and Synth. Commun. 20(22): 3537-3545 (1990). The second stage is a conventional reduction stage, and can be, for example, a hydrogenation reaction by heterogeneous catalysis in the presence of a catalyst chosen from Pd/C, Pd(II)/C, and Raney Nickel, or a reduction reaction by a metal, for example, zinc, iron, tin, and the like (see, for example, J. March, Advanced Organic Chemistry, 4^{th} ed., 1992, Wiley lnterscience; M. Hudlicky, Reduction in Organic Chemistry, 1983, Ellis Horwood Series Chemical Science).

Intermediate Compounds of Formula (II)

Also disclosed herein are nitrogen-containing compounds of formula (II) which can be intermediate compounds used for obtaining para-phenylenediamines of formula (I): 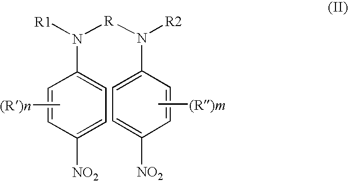
wherein R₁, R₂, R, R′, R″, n, and m are defined above in the context of formula (I), with the proviso that the compound of formula (II) is not 1,2-propanediamine N,N′-bis(4-nitrophenyl).
Dyeing Compositions

Further disclosed herein is a dyeing composition comprising, in a medium suitable for dyeing, at least one oxidation base chosen from para-phenylenediamine compounds of formula (I) and the addition salts thereof.

The at least one oxidation base chosen from para-phenylenediamines of formula (I) and the addition salts thereof can be present in the dyeing composition in an amount ranging from 0.0001 wt. % to 20 wt. % relative to the total weight of the dyeing composition, for example, from 0.01 wt. % to 10 wt. %.

Oxidation Couplers

In at least one embodiment, the composition of the present disclosure may comprise at least one oxidation coupler.

Examples of suitable oxidation couplers include, but are not limited to, meta-phenylenediamines, meta-aminophenols, meta-diphenols, naphthalenic couplers, heterocyclic couplers, and the addition salts thereof.

Further non-limiting examples of oxidation couplers include: 2-methyl-5-aminophenol, 5-N-(β-hydroxyethyl)amino-2-methylphenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diamino-benzene, 1,3-bis-(2,4-diaminophenoxy)propane, 3-ureido-aniline, 3-ureido-1-dimethylaminobenzene, sesamol, 1-β-hydroxyethylamino-3,4-methylene-dioxybenzene, α-naphthol, 2-methyl-1-naphthol, 6-hydroxy-indole, 4-hydroxy-indole, 4-hydroxy-N-methyl indole, 2-amino-3-hydroxypyridine, 6-hydroxybenzomorpholine, 3,5-diamino-2,6-dimethoxypyridine, 1-N-(β-hydroxyethyl)amino-3,4-methylene dioxybenzene, 2,6-bis-(β-hydroxyethylamino)toluene, and the addition salts thereof.

The at least one oxidation coupler may be present in the dyeing composition in an amount ranging from 0.0001 to 20 wt. %, for example, from 0.005 to 6 wt. %, relative to the total weight of the dyeing composition.

Additional Oxidation Bases

According to another embodiment, the composition of the present disclosure may also comprise at least one additional oxidation base different from the oxidation bases of formula (I).

The at least one additional oxidation base may be chosen, for example, from para-phenylenediamines, bis-phenylalkylenediamines, para-aminophenols, ortho-aminophenols, heterocyclic bases, and the addition salts thereof.

Examples of suitable para-phenylenediamines include, but are not limited to: para-phenylenediamine, para-toluenediamine, 2-chloro-para-phenylenediamine, 2,3-dimethyl para-phenylenediamine, 2,6-dimethyl para-phenylenediamine, 2,6-diethyl para-phenylenediamine, 2,5-dimethyl para-phenylenediamine, N,N-dimethyl para-phenylenediamine, N,N-diethyl para-phenylenediamine, N,N-dipropyl para-phenylenediamine, 4-amino-N,N-diethyl-3-methyl aniline, N,N-bis-(β-hydroxyethyl)para-phenylenediamine, 4-N,N-bis-(β-hydroxyethyl)amino-2-methyl aniline, 4-N,N-bis(β-hydroxyethyl)amino-2-chloro-aniline, 2-β-hydroxyethyl para-phenylenediamine, 2-fluoro para-phenylenediamine, 2-isopropyl para-phenylenediamine, N-(β-hydroxypropyl)para-phenylenediamine, 2-hydroxymethyl para-phenylenediamine, N,N-dimethyl-3-methyl para-phenylenediamine, N,N-(ethyl, β-hydroxyethyl)para-phenylenediamine, N-(β,γ-dihydroxypropyl) para-phenylenediamine, N-(4′-aminophenyl)para-phenylenediamine, N-phenyl para-phenylenediamine, 2-β-hydroxyethyloxy para-phenylenediamine, 2-β-acetylaminoethyloxy para-phenylenediamine, N-(β-methoxyethyl)para-phenylenediamine, 2-thienyl para-phenylenediamine, 2-β-hydroxyethylamino-5-aminotoluene, 3-hydroxy-1-(4′-aminophenyl)pyrrolidone, 6-(4-amino-phenylamino)-hexan-1-ol, and the addition salts thereof.

In at least one embodiment, the at least one additional oxidation base may be a para-phenylenediamine chosen from para-phenylenediamine, para-toluenediamine, 2-isopropyl para-phenylenediamine, 2-β-hydroxyethyl para-phenylenediamine, 2-β-hydroxyethyloxy para-phenylenediamine, 2,6-dimethyl para-phenylenediamine, 2,6-diethyl para-phenylenediamine, 2,3-dimethyl para-phenylenediamine, N,N-bis-(β-hydroxyethyl)para-phenylenediamine, 2-chloro para-phenylenediamine, 2-β-acetylaminoethyloxy para-phenylenediamine, 6-(4-amino-phenylamino)-hexan-1-ol, and the acid addition salts thereof.

Non-limiting examples of bis-phenylalkylenediamines include: N,N′-bis-(β-hydroxyethyl) N,N′-bis-(4′-aminophenyl) 1,3-diamino-propanol, N,N′-bis(β-hydroxyethyl) N,N′-bis(4′-aminophenyl) ethylenediamine, N,N′-bis-(4′-aminophenyl)tetramethylenediamine, N,N′-bis-(β-hydroxyethyl) N,N′-bis-(4′-aminophenyl)tetramethylenediamine, N,N′-bis-(4′-amino-3′-methylphenyl)tetramethylenediamine, N,N′-bis-(ethyl) N,N′-bis-(4′-amino, 3′-methylphenyl) ethylenediamine, 1,8-bis-(2,5-diamino-phenoxy)-3,6-dioxaoctane, and the acid addition salts thereof.

Suitable para-aminophenols include, by way of non-limiting example: para-aminophenol, 4-amino-3-methylphenol, 4-amino-3-fluorophenol, 4-amino-2-chlorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethyl aminomethyl)phenol, 4-amino-2-fluorophenol, 4-amino-2,6-dichlorophenol, 4-amino-6[((5′-amino-2′-hydroxy-3′-methyl)phenyl)methyl]-2-methylphenol, bis(5′-amino-2′-hydroxy)phenylmethane, and the acid addition salts thereof.

Examples of ortho-aminophenols include, but are not limited to: 2-aminophenol, 2-amino-5-methylphenol, 2-amino-6-methylphenol, 5-acetamido-2-aminophenol, 5-[(2-hydroxyethyl)amino]2-methylphenol, and the acid addition salts thereof.

Heterocyclic bases suitable for use in the compositions of the present disclosure include, for example: pyridine derivatives, pyrimidine derivatives, and pyrazole derivatives.

Non-limiting examples of pyridine derivatives include the compounds described in British Patent Nos. 1 026 978 and 1 153 196, such as 2,5-diamino pyridine, 2-(4-methoxyphenyl)amino-3-amino pyridine, 3,4-diamino pyridine, and the acid addition salts thereof.

Suitable pyridine oxidation bases include, for example, 3-amino pyrazolo-[1,5-a]-pyridines and the addition salts thereof. These compounds are described, for example, in French Patent Application No. 2 801 308, for instance, pyrazolo[1,5-a]pyridin-3-ylamine; 2-acetylamino pyrazolo-[1,5-a]pyridin-3-ylamine; 2-morpholin-4-yl-pyrazolo[1,5-a]pyridin-3-ylamine; 3-amino-pyrazolo[1,5-a]pyridine-2-carboxylic acid; 2-methoxy-pyrazolo[1,5-a]pyridin-3-ylamine; (3-amino-pyrazolo[1,5-a]pyridin-7-yl)-methanol; 2-(3-amino-pyrazolo[1,5-a]pyridin-5-yl)-ethanol; 2-(3-amino-pyrazolo[1,5-a]pyridin-7-yl)-ethanol; (3-amino -pyrazolo[1,5-a]pyridin-2-yl)-methanol; 3,6-diamino-pyrazolo[1,5-a]pyridine; 3,4-diamino -pyrazolo[1,5-a]pyridine; pyrazolo[1,5-a]pyridine-3,7-diamine; 7-morpholin-4-yl-pyrazolo[1,5-a]pyridin-3-ylamine; pyrazolo[1,5-a]pyridine-3,5-diamine; 5-morpholin-4-yl-pyrazolo[1,5-a] pyridin-3-ylamine; 2-[(3-amino-pyrazolo[1,5-a]pyridin-5-yl)-(2-hydroxyethyl)-amino]-ethanol; 2-[(3-amino-pyrazolo[1,5-a]pyridin-7-yl)-(2-hydroxyethyl)-amino]-ethanol; 3-amino -pyrazolo[1,5-a]pyridin-5-ol; 3-amino-pyrazolo[1,5-a]pyridin-4-ol; 3-amino-pyrazolo[1,5-a]pyridin-6-ol; 3-amino-pyrazolo[1,5-a]pyridin-7-ol; and the acid addition salts thereof.

Examples of pyrimidine derivatives include, but are not limited to, the compounds described in German Patent No. 23 59 399; Japanese Patent Application No. 88-169571; Japanese Patent No. 5-63124; European Patent No. 0 770 375; and International Patent Application Publication No. WO 96/15765, such as 2,4,5,6-tetra-aminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, the addition salts thereof and the tautomeric forms thereof, when a tautomeric equilibrium exists.

Pyrazole derivatives suitable for use in the composition of the present disclosure may include, for example, the compounds described in German Patent Nos. 38 43 892 and 41 33 957, International Patent Application Publication Nos. WO 94/08969 and WO 94/08970, French Patent Application No. 2 733 749, and German Patent Application No. 195 43 988, such as 4,5-diamino-1-methyl pyrazole, 4,5-diamino-1-(β-hydroxyethyl) pyrazole, 3,4-diamino pyrazole, 4,5-diamino-1-(4′-chlorobenzyl) pyrazole, 4,5-diamino-1,3-dimethyl pyrazole, 4,5-diamino-3-methyl-1-phenyl pyrazole, 4,5-diamino-1-methyl-3-phenyl pyrazole, 4-amino-1,3-dimethyl-5-hydrazino pyrazole, 1-benzyl-4,5-diamino-3-methyl pyrazole, 4,5-diamino-3-tert-butyl 1-methyl pyrazole, 4,5-diamino-1-tert-butyl 3-methyl pyrazole, 4,5-diamino-1-(β-hydroxyethyl)-3-methyl pyrazole, 4,5-diamino-1-ethyl-3-methyl pyrazole, 4,5-diamino-1-ethyl-3-(4′-methoxyphenyl) pyrazole, 4,5-diamino-1-ethyl-3-hydroxymethyl pyrazole, 4,5-diamino-3-hydroxymethyl-1-methyl pyrazole, 4,5-diamino-3-hydroxymethyl-1-isopropyl pyrazole, 4,5-diamino-3-methyl-1-isopropyl pyrazole, 4-amino-5-(2′-aminoethyl)amino-1,3-dimethyl pyrazole, 3,4,5-triamino pyrazole, 1-methyl -3,4,5-triamino pyrazole, 3,5-diamino-1-methyl-4-methylamino pyrazole, 3,5-diamino-4-(β-hydroxyethyl)amino-1-methyl pyrazole, and the addition salts thereof.

The at least one additional oxidation base may be present in the dyeing composition in an amount ranging from 0.0001 to 20 wt. %, for example, from 0.005 to 6 wt. %, relative to the total weight of the dyeing composition.

Direct Dyes

The dyeing composition of the present disclosure may further comprise at least one direct dye chosen, for example, from neutral, acidic, and cationic nitrogen-containing dyes of the benzene series; neutral, acidic, and cationic direct azo dyes; neutral, acidic, and cationic quinone, for instance, anthraquinone, direct dyes; azine direct dyes; methine direct dyes; azomethine direct dyes; triarylmethane direct dyes; indoamine direct dyes; and natural direct dyes. In one embodiment, the composition of the present disclosure may comprise at least one dye chosen from cationic direct dyes and natural direct dyes.

Examples of cationic direct dyes include, but are not limited to, cationic azo direct dyes described, for example, in International Patent Application Publication Nos. WO 95/15144 and WO-95/01772 and European Patent Application No. 0 714 954.

These compounds may include, for example:
1,3-dimethyl-2-[[4-(dimethylamino)phenyl]azo]-1H-imidazolium chloride,
1,3-dimethyl-2-[(4-aminophenyl)azo]-1H-imidazolium chloride, and
1-methyl-4-[(methylphenylhydrazono)methyl]-pyridinium methylsulphate.

Natural direct dyes suitable for use in accordance with the present disclosure may include, for example, lawsone, juglone, alizarin, purpurin, carminic acid, kermesic acid, purpurogallin, protocatechaldehyde, indigo, isatin, curcumine, spinulosine, and apigenidine. In at least one embodiment, extracts or decoctions comprising these natural dyes may be used, for instance, henna-based cataplasms or extracts.

The at least one direct dye may be present in the composition in an amount ranging from 0.001 to 20 wt. %, relative to the total weight of the composition, for example, from 0.005 to 10 wt. %.

Dyeing Medium

The medium suitable for dyeing may be chosen from water and mixtures of water and at least one organic solvent, for example, linear and branched C₁-C₄ lower alcohols, such as ethanol and isopropanol; polyols and polyol ethers, such as 2-butoxyethanol, propyleneglycol, monomethyl ether of propyleneglycol, monoethyl ether of diethyleneglycol, monomethyl ether of diethyleneglycol, and glycerol; aromatic alcohols, such as benzyl alcohol and phenoxyethanol; and mixtures thereof.

The at least one solvent may be present in the dyeing composition in an amount ranging from 1 to 40 wt. % relative to the total weight of the dyeing composition, for example, from 5 to 30 wt. %.

Additives

The dyeing composition may also comprise at least one cosmetic additive chosen from antioxidants, penetrants, sequestering agents, perfumes, buffers, dispersants, surfactants, conditioners, film-forming agents, polymers, ceramides, preservatives, lustre agents, opacifiers, vitamins and provitamins.

The at least one additive may be present in the dyeing composition in an amount, for each of them, ranging from 0.01 to 20 wt. % relative to the total weight of the dyeing composition.

The pH of the composition according to the present disclosure may range from 3 to 12, for example, from 5 to 11. The pH may be adjusted to a desired value by means of acidifying or alkalizing agents conventionally employed in the dyeing of keratin fibers or by means of conventional buffering systems.

Examples of acidifying agents include, for example, inorganic or organic acids other than dicarboxylic acids, such as hydrochloric acid, orthophosphoric acid, sulphuric acid, carboxylic acids such as acetic acid, tartaric acid, citric acid, lactic acid, and sulphonic acids.

Alkalizing agents may be chosen, for example, from: ammonia, alkali metal carbonates, alkanolamines such as mono-, di-, and triethanolamines and their derivatives, hydroxides of sodium, hydroxides of potassium, and compounds of formula (III): 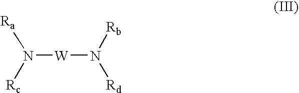
wherein:
W is chosen from propylene residues optionally substituted by at least one entity chosen from hydroxyl groups and C₁-C₄ alkyl radicals; and
Rₐ, R_{b}, R_{c}, and R_{d}, which may be identical or different, are chosen from hydrogen, C₁-C₄ alkyl radicals, and C₁-C₄ hydroxyalkyl radicals.

It is to be understood that a person skilled in the art will ensure that any envisaged optional additions, for example, addition of at least one additive, at least one precursor of additional oxidation dyes, at least one oxidation coupler, and/or at least one direct dye, are chosen in such a way that the beneficial properties intrinsically associated with the oxidation dyeing composition according to the present disclosure are not, or are not substantially, adversely affected.

The dyeing composition according to the present disclosure may be in various forms, such as liquids, creams, and gels, or in any other form suitable for dyeing keratin fibers, for example, human hair.

Method for Dyeing

Further disclosed herein is a method for dyeing keratin fibers comprising applying a composition of the present disclosure to the fibers, and developing the color by means of at least one oxidizing agent. The color may be developed at acidic, neutral, or alkaline pH. In one embodiment, the at least one oxidizing agent may be added to the composition of the present disclosure at the moment of use. According to another embodiment, the at least one oxidizing agent it may be applied via an oxidizing composition comprising it, which may be applied simultaneously or sequentially with the composition of the present disclosure.

Examples of suitable oxidizing agents include, but are not limited to, hydrogen peroxide; urea peroxide; bromates of alkali metals; persalts, such as perborates and persulphates; peracids; and oxidase enzymes, such as peroxidases, 2-electron oxido-reductases, such as the uricases, and 4-electron oxygenases, such as the laccases. In at least one embodiment, the at least one oxidizing agent is hydrogen peroxide.

According to one embodiment, the composition of the present disclosure may be mixed, for instance, at the moment of use, with a composition comprising, in a medium suitable for dyeing, at least one oxidizing agent, this oxidizing agent being present in a sufficient amount for dye development. The mixture obtained may then be applied to the keratin fibers. After a period of coloration time ranging from 3 to 50 minutes, for example, from 5 to 30 minutes, the keratin fibers may be rinsed, washed with shampoo, rinsed again, and then dried.

The oxidizing composition may comprise at least one additive among the various additives used conventionally in compositions for hair dyeing and as defined above.

The pH of the oxidizing composition comprising the oxidizing agent may be such that, after mixing with the dyeing composition, the pH of the resulting composition applied to the keratin fibers ranges from 3 to 12, for instance, from 5 to 11. The pH may be adjusted to a desired value by means of acidifying or alkalizing agents conventionally employed in the dyeing of keratin fibers and as defined above.

The ready-to-use composition that is applied to the keratin fibers may be in various forms, such as liquids, creams, and gels, or in any other form suitable for dyeing keratin fibers, for example human keratin fibers such as the hair.

The present disclosure also relates to the use of the cosmetic composition as disclosed herein comprising, in a medium that is suitable for dyeing, at least one para-phenylenediamine of formula (I) for the dyeing of keratin fibers, including human keratin fibers such as the hair.

Multi-compartment Device

Further disclosed herein is a multi-compartment device or dyeing “kit” comprising at least one first compartment containing at least one dyeing composition comprising of the present disclosure, and at least one second compartment containing at least one oxidizing composition. This kit may be equipped with an applicator for delivering the desired mixture onto the hair, such as the kits described in French Patent No. 2 586 913.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, unless otherwise indicated, the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLES

### Synthesis Examples

### Example 1

### Synthesis of 2,2-dimethyl-N,N′-bis(4-aminophenyl)propane-1,3-diamine tetrahydrochloride (2)

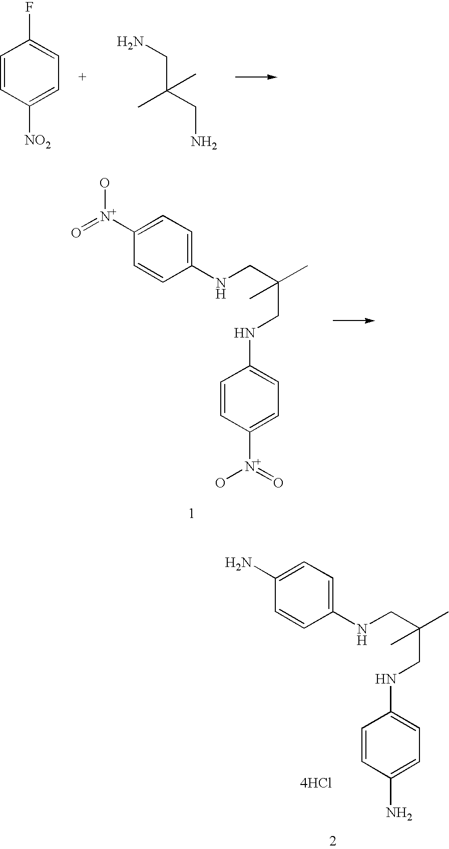 Synthesis of 2,2-dimethyl-N,N′-bis(4-nitrophenyl)propane-1,3-diamine (1)

2.3 g of 4-fluoronitrobenzene (2 eq.) were dissolved in 10 ml of DMSO. 1.2 equivalents of 2,2-dimethyl-1,3-propanediamine and 4 equivalents of triethylamine were added to the solution. The reaction mixture was heated at 60° C. for 20 hours. The mixture was then poured onto crushed ice; a precipitate formed. The latter was filtered, washed with water, and then dried.

Synthesis of 2,2-dimethyl-N,N′-bis(4-aminophenyl)propane-1,3-diamine tetrahydrochloride (2)

The 2,2-dimethyl-N,N′-bis(4-nitrophenyl)propane-1,3-diamine (1) obtained in the preceding stage was reduced with a boiling zinc/ammonium chloride/water/ethanol mixture. The corresponding amine was isolated in the form of the tetrahydrochloride.

The proton and mass NMR spectra corresponded to the expected structure of the product.

### Example 2

### Synthesis of 2,2-dimethyl-N,N′-bis(3-methyl-4-aminophenyl)propane-1,3-diamine tetrahydrochloride (4)

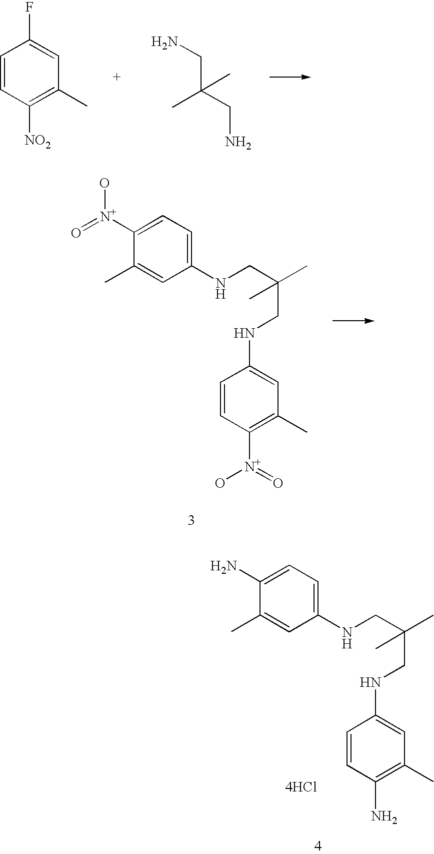 Synthesis of 2,2-dimethyl-N,N′-bis(3-methyl-4-nitrophenyl)propane-1,3-diamine (3)

1.5 g of 5-fluoro-2-nitrotoluene (2 eq.) were dissolved in 10 ml of DMSO. 1.2 equivalents of 2,2-dimethyl-1,3-propanediamine and 1.2 equivalents of triethylamine were added to the solution. The reaction mixture was heated at 80° C. for 24 hours. The mixture was then poured onto crushed ice; a precipitate formed. The latter was filtered, washed with water, and then dried.

Synthesis of 2,2-dimethyl-N,N′-bis(3-methyl-4-aminophenyl)propane-1,3-diamine tetrahydrochloride (4)

The 2,2-dimethyl-N,N′-bis(3-methyl-4-nitrophenyl)propane-1,3-diamine (3) obtained in the preceding stage was reduced with a boiling zinc/ammonium chloride/water/ethanol mixture. The corresponding amine was isolated in the form of the tetrahydrochloride.

The proton and mass NMR spectra corresponded to the expected structure of the product.

### Example 3

### Synthesis of 2,2-dimethyl-N,N′-bis(2-methyl-4-amino-phenyl)propane-1,3-diamine tetrahydrochloride (6)

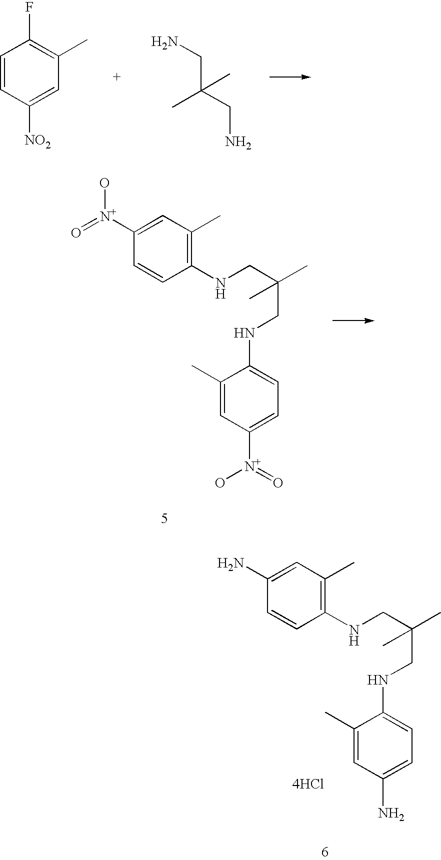 Synthesis of 2,2-dimethyl-N,N′-bis(2-methyl-4-nitrophenyl)propane-1,3-diamine (5)

1.5 g of 2-fluoro-5-nitrotoluene (2 eq.) were dissolved in 10 ml of DMSO. 1.2 equivalents of 2,2-dimethyl-1,3-propanediamine and 1.2 equivalents of triethylamine were added to the solution. The reaction mixture was heated at 80° C. for 24 hours. The mixture was then poured onto crushed ice; a precipitate formed. The latter was filtered, washed with water, and then dried.

Synthesis of 2,2-dimethyl-N,N′-bis(2-methyl-4-aminophenyl)propane-1,3-diamine tetrahydrochloride (6)

The 2,2-dimethyl-N,N′-bis(2-methyl-4-nitrophenyl)propane-1,3-diamine (5) obtained in the preceding stage was reduced with a boiling zinc/ammonium chloride/water/ethanol mixture. The corresponding amine was isolated in the form of the tetrahydrochloride.

The proton and mass NMR spectra corresponded to the expected structure of the product.

### Example 4

### Synthesis of N-{3-[(4-aminophenyl)amino]-1-ethylpropyl}benzene-1,4-diamine tetrahydrochloride (8)

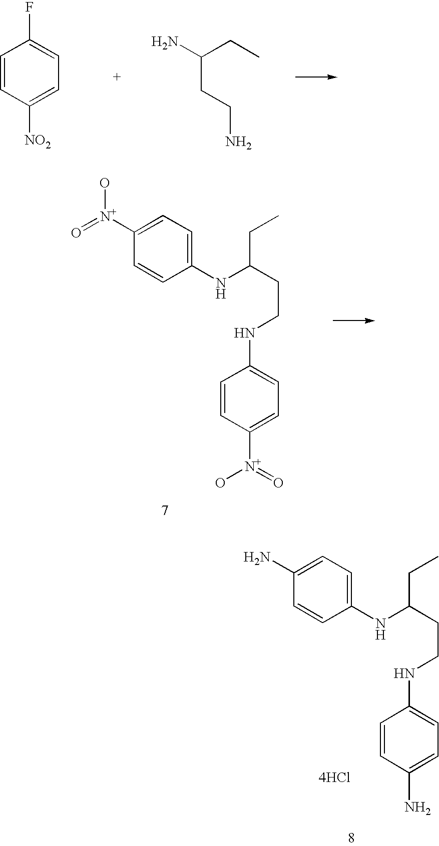 Synthesis of N,N′-bis(4-nitrophenyl)pentane-1,3-diamine (7)

2.3 g of 4-fluoronitrobenzene (2 eq.) were dissolved in 10 ml of DMSO. 1.2 equivalents of 3-amino-1-ethylpropylamine and 4 equivalents of triethylamine were added to the solution. The reaction mixture was heated at 60° C. for 16 hours. The mixture was then poured onto crushed ice; a precipitate formed. The latter was filtered, washed with water, and then dried.

Synthesis of N-{3-[(4-aminophenyl)amino]-1-ethylpropyl}benzene-1,4-diamine tetrahydrochloride (8)

The N,N′-bis(4-nitrophenyl)pentane-1,3-diamine (7) obtained in the preceding stage was reduced with a boiling zinc/ammonium chloride/water/ethanol mixture. The corresponding amine was isolated in the form of the tetrahydrochloride.

The proton and mass NMR spectra corresponded to the expected structure of the product.

### Example 5

### Synthesis of N-(4-aminophenyl)-N-{5-[(4-aminophenyl)amino]-2-methylpentyl}amine tetrahydrochloride (10)

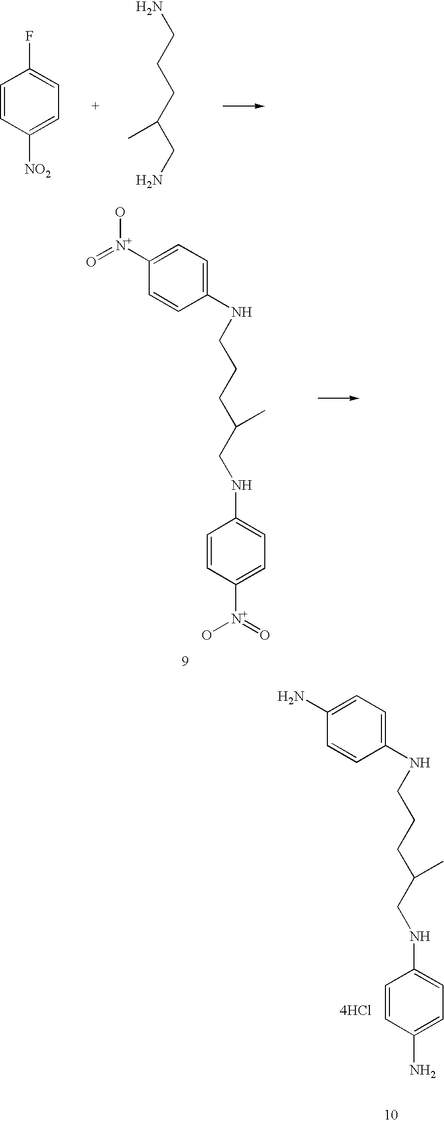 Synthesis of N-{2-methyl-5-[(4-nitrophenyl)amino]pentyl}-N-(4-nitrophenyl)amine (9)

2.3 g of 4-fluoronitrobenzene (2 eq.) were dissolved in 10 ml of DMSO. 1.2 equivalents of 2-methylpentane-1,5-diamine and 4 equivalents of triethylamine were added to the solution. The reaction mixture was heated at 60° C. for 20 hours. The mixture was then poured onto crushed ice; a precipitate formed. The latter was filtered, washed with water, and then dried.

Synthesis of N-(4-aminophenyl)-N-{5-[(4-aminophenyl)amino]-2-methylpentyl}amine tetrahydrochloride (10)

The N-{2-methyl-5-[(4-nitrophenyl)amino]pentyl}-N-(4-nitrophenyl)amine obtained in the preceding stage was reduced with a boiling zinc/ammonium chloride/water/ethanol mixture. The corresponding amine was isolated in the form of the tetrahydrochloride.

The proton and mass NMR spectra corresponded to the expected structure of the product.

### EXAMPLES OF DYEING COMPOSITIONS

### Examples 1 to 14

### Dyeing Composition Based on 2,2-dimethyl-N,N′-bis(4-aminophenyl)propane-1,3-diamine tetrahydrochloride (2)

### Examples 1 to 7

### Dyeing in an Acid Medium

The following dyeing compositions were prepared:

| | Example | | | | | | |
|---|---|---|---|---|---|---|---|
| | 1 | 2 | 3 | 4 | 5 | 6 | 7 |
| 2,2-dimethyl-N,N′-bis(4- | 10⁻³ mol | 10⁻³ mol | 10⁻³ mol | 10⁻³ mol | 10⁻³ mol | 10⁻³ mol | 10⁻³ mol |
| aminophenyl)propane-1,3-diamine | | | | | | | |
| tetrahydrochloride (2) | | | | | | | |
| Benzene-1,3-diol | 10⁻³ mol | | | | | | |
| 5-Amino-2-methyl-phenol | | 10⁻³ mol | | | | | |
| 1H-Indol-6-ol | | | 10⁻³ mol | | | | |
| 2-Amino-pyridin-3-ol | | | | 10⁻³ mol | | | |
| 3,6-Dimethyl-1H-pyrazolo | | | | | 10⁻³ mol | | |
| [5,1-c][1,2,4]triazole | | | | | | | |
| 2-(2,4-Diamino-phenoxy)- | | | | | | 10⁻³ mol | |
| ethanol hydrochloride | | | | | | | |
| 3-Amino-2-chloro-6-methyl- | | | | | | | 10⁻³ mol |
| phenol hydrochloride | | | | | | | |
| Dyeing support (1) | (*) | (*) | (*) | (*) | (*) | (*) | (*) |
| Demineralized water q.s.f. | 100 g | 100 g | 100 g | 100 g | 100 g | 100 g | 100 g |

(*): dyeing support (1) pH 7

| | | |
|---|---|---|
| 96° ethanol | 20.8 | g |
| Sodium metabisulphite, 35% aqueous solution | 0.23 | g M.A |
| Pentasodium salt of diethylene-triamine-pentaacetic | 0.48 | g M.A |
| acid, 40% aqueous solution | | |
| C₈-C₁₀ alkyl polyglycoside, 60% aqueous solution | 3.6 | g M.A |
| Benzyl alcohol | 2.0 | g |
| Polyethylene glycol with 8 ethylene oxide units | 3.0 | g |
| Na₂HPO₄ | 0.28 | g |
| KH₂PO₄ | 0.46 | g |

At the time of use, each composition was mixed with an equal weight of hydrogen peroxide at 20 volumes (6 wt. %). A final pH of 7 was obtained.

Each mixture obtained was applied to locks of grey hair comprising 90% white hairs. After a holding time of 30 minutes, the locks were rinsed, washed with a standard shampoo, rinsed again, and then dried.

The shades obtained are shown in the following table:

| | Example | | | | | | |
|---|---|---|---|---|---|---|---|
| | 1 | 2 | 3 | 4 | 5 | 6 | 7 |
| Shade | brown | deep | deep | deep | red- | deep | deep |
| observed | | grey | brown | red- | brown | blue | violet |
| | | violet- | | brown | | | |
| | | red | | | | | |

### Examples 8 to 14

### Dyeing in a Basic Medium

The following dyeing compositions were prepared:

| | Example | | | | | | |
|---|---|---|---|---|---|---|---|
| | 8 | 9 | 10 | 11 | 12 | 13 | 14 |
| 2,2-dimethyl-N,N′-bis(4- | 10⁻³ mol | 10⁻³ mol | 10⁻³ mol | 10⁻³ mol | 10⁻³ mol | 10⁻³ mol | 10⁻³ mol |
| aminophenyl)propane-1,3- | | | | | | | |
| diamine tetrahydrochloride | | | | | | | |
| (2) | | | | | | | |
| Benzene-1,3-diol | 10⁻³ mol | | | | | | |
| 5-Amino-2-methyl-phenol | | 10⁻³ mol | | | | | |
| 1H-Indol-6-ol | | | 10⁻³ mol | | | | |
| 2-Amino-pyridin-3-ol | | | | 10⁻³ mol | | | |
| 3,6-Dimethyl-1H-pyrazolo | | | | | 10⁻³ mol | | |
| [5,1-c][1,2,4]triazole | | | | | | | |
| 2-(2,4-Diamino-phenoxy)- | | | | | | 10⁻³ mol | |
| ethanol hydrochloride | | | | | | | |
| 3-Amino-2-chloro-6-methyl- | | | | | | | 10⁻³ mol |
| phenol hydrochloride | | | | | | | |
| Dyeing support (2) | (*) | (*) | (*) | (*) | (*) | (*) | (*) |
| Demineralized water q.s.f. | 100 g | 100 g | 100 g | 100 g | 100 g | 100 g | 100 g |

(*): dyeing support (2) pH 9.5

| | | |
|---|---|---|
| 96° ethanol | 20.8 | g |
| Sodium metabisulphite, 35% aqueous solution | 0.23 | g M.A |
| Pentasodium salt of diethylene-triamine-pentaacetic | 0.48 | g M.A |
| acid, 40% aqueous solution | | |
| C₈-C₁₀ alkyl polyglycoside, 60% aqueous solution | 3.6 | g M.A |
| Benzyl alcohol | 2.0 | g |
| Polyethylene glycol with 8 ethylene oxide units | 3.0 | g |
| NH₄Cl | 4.32 | g |
| Ammonia at 20% NH₃ | 2.94 | g |

At the time of use, each composition was mixed with an equal weight of hydrogen peroxide at 20 volumes (6 wt. %). A final pH of 9.5 was obtained.

Each mixture obtained was applied to locks of grey hair comprising 90% white hairs. After a period of coloration time of 30 minutes, the locks were rinsed, washed with a standard shampoo, rinsed again, and then dried.

The shades obtained are shown in the following table:

| | Example | | | | | | |
|---|---|---|---|---|---|---|---|
| | 8 | 9 | 10 | 11 | 12 | 13 | 14 |
| Shade | brown | deep | deep | deep | deep | deep | deep |
| observed | | violet- | red | red- | red- | blue | violet |
| | | grey | | brown | violet | | |

### Examples 15 to 25

### Dyeing Composition Based on 2,2-dimethyl-N,N′-bis(3-methyl-4-aminophenyl)propane-1,3-diamine tetrahydrochloride (4)

### Examples 15 to 19

### Dyeing in an Acid Medium

The following dyeing compositions were prepared:

| Example | 15 | 16 | 17 | 18 | 19 |
|---|---|---|---|---|---|
| 2,2-dimethyl-N,N′-bis(3-methyl-4- | 10⁻³ | 10⁻³ | 10⁻³ | 10⁻³ | 10⁻³ |
| aminophenyl)propane-1,3-diamine | mol | mol | mol | mol | mol |
| tetrahydrochloride (4) | | | | | |
| 5-Amino-2-methyl-phenol | 10⁻³ | | | | |
| | mol | | | | |
| 1H-indol-6-ol | | 10⁻³ | | | |
| | | mol | | | |
| 2-Amino-pyridin-3-ol | | | 10⁻³ | | |
| | | | mol | | |
| 2-(2,4-Diamino-phenoxy)-ethanol | | | | 10⁻³ | |
| hydrochloride | | | | mol | |
| 3-Amino-2-chloro-6-methyl-phenol | | | | | 10⁻³ |
| hydrochloride | | | | | mol |
| Dyeing support (1) | (*) | (*) | (*) | (*) | (*) |
| Demineralized water q.s.f. | 100 g | 100 g | 100 g | 100 g | 100 g |

(*): dyeing support (1) as described above

At the time of use, each composition was mixed with an equal weight of hydrogen peroxide at 20 volumes (6 wt. %). A final pH of 7 was obtained.

Each mixture obtained was applied to locks of grey hair comprising 90% white hairs. After a period of coloration time of 30 minutes, the locks were rinsed, washed with a standard shampoo, rinsed again, and then dried.

The shades obtained are shown in the following table:

Example

| | 15 | 16 | 17 | 18 | 19 |
|---|---|---|---|---|---|
| Shade | violet- | brown | yellow | deep | deep |
| observed | grey | | | blue | violet- |
| | | | | | blue |

### Examples 20 to 25

### Dyeing in a Basic medium

The following dyeing compositions were prepared:

| | Example | | | | | |
|---|---|---|---|---|---|---|
| | 20 | 21 | 22 | 23 | 24 | 25 |
| 2,2-dimethyl-N,N′-bis(3-methyl-4- | 10⁻³ mol | 10⁻³ mol | 10⁻³ mol | 10⁻³ mol | 10⁻³ mol | 10⁻³ mol |
| aminophenyl)propane-1,3-diamine | | | | | | |
| tetrahydrochloride (4) | | | | | | |
| 5-Amino-2-methyl-phenol | 10⁻³ mol | | | | | |
| 1H-Indol-6-ol | | 10⁻³ mol | | | | |
| 2-Amino-pyridin-3-ol | | | 10⁻³ mol | | | |
| 3,6-Dimethyl-1H-pyrazolo | | | | 10⁻³ mol | | |
| [5,1-c][1,2,4]triazole | | | | | | |
| 2-(2,4-Diamino-phenoxy)-ethanol | | | | | 10⁻³ mol | |
| hydrochloride | | | | | | |
| 3-Amino-2-chloro-6-methyl-phenol | | | | | | 10⁻³ mol |
| hydrochloride | | | | | | |
| Dyeing support (2) | (*) | (*) | (*) | (*) | (*) | (*) |
| Demineralized water q.s.f. | 100 g | 100 g | 100 g | 100 g | 100 g | 100 g |

(*): dyeing support (2) as described above

At the moment of use, each composition was mixed with an equal weight of hydrogen peroxide at 20 volumes (6 wt. %). A final pH of 9.5 was obtained.

Each mixture obtained was applied to locks of grey hair comprising 90% white hairs. After a period of coloration time of 30 minutes, the locks were rinsed, washed with a standard shampoo, rinsed again, and then dried.

The shades obtained are shown in the following table:

Example

| | 20 | 21 | 22 | 23 | 24 | 25 |
|---|---|---|---|---|---|---|
| Shade | deep | red | orangey | red | deep | grey |
| observed | violet | | | | blue | violet- |
| | | | | | | blue |

### Examples 26 to 36

### Dyeing Composition Based on 2,2-dimethyl-N,N′-bis(2-methyl-4-aminophenyl)propane-1,3-diamine tetrahydrochloride (6)

### Examples 26 to 30

### Dyeing in an Acidic Medium

The following dyeing compositions were prepared:

| | Example | | | | |
|---|---|---|---|---|---|
| | 26 | 27 | 28 | 29 | 30 |
| 2,2-dimethyl-N,N′-bis(2-methyl-4- | 10⁻³ mol | 10⁻³ mol | 10⁻³ mol | 10⁻³ mol | 10⁻³ mol |
| aminophenyl)propane-1,3-diamine | | | | | |
| tetrahydrochloride (6) | | | | | |
| 5-Amino-2-methyl-phenol | 10⁻³ mol | | | | |
| 1H-Indol-6-ol | | 10⁻³ mol | | | |
| 2-Amino-pyridin-3-ol | | | 10⁻³ mol | | |
| 2-(2,4-Diaminophenoxy)-ethanol | | | | 10⁻³ mol | |
| hydrochloride | | | | | |
| 3-Amino-2-chloro-6-methyl-phenol | | | | | 10⁻³ mol |
| hydrochloride | | | | | |
| Dyeing support (1) | (*) | (*) | (*) | (*) | (*) |
| Demineralized water q.s.f. | 100 g | 100 g | 100 g | 100 g | 100 g |

(*): dyeing support (1) as described above

At the time of use, each composition was mixed with an equal weight of hydrogen peroxide at 20 volumes (6 wt. %). A final pH of 7 was obtained.

Each mixture obtained was applied to locks of grey hair comprising 90% white hairs. After a period of coloration time of 30 minutes, the locks were rinsed, washed with a standard shampoo, rinsed again, and then dried.

The shades obtained are shown in the following table:

Example

| | 26 | 27 | 28 | 29 | 30 |
|---|---|---|---|---|---|
| Shade | red- | deep | orangey | deep | deep |
| observed | violet | grey | brown | blue | violet |
| | grey | | | | |

### Examples 31 to 35

### Dyeing in a Basic Medium

The following dyeing compositions were prepared:

| | Example | | | | |
|---|---|---|---|---|---|
| | 31 | 32 | 33 | 34 | 35 |
| 2,2-dimethyl-N,N′-bis(2-methyl-4- | 10⁻³ mol | 10⁻³ mol | 10⁻³ mol | 10⁻³ mol | 10⁻³ mol |
| aminophenyl)propane-1,3-diamine | | | | | |
| tetrahydrochloride (6) | | | | | |
| 5-Amino-2-methyl-phenol | 10⁻³ mol | | | | |
| 1H-Indol-6-ol | | 10⁻³ mol | | | |
| 2-Amino-pyridin−3-ol | | | 10⁻³ mol | | |
| 2-(2,4-Diaminophenoxy)-ethanol | | | | 10⁻³ mol | |
| hydrochloride | | | | | |
| 3-Amino-2-chloro-6-methyl-phenol | | | | | 10⁻³ mol |
| hydrochloride | | | | | |
| Dyeing support (2) | (*) | (*) | (*) | (*) | (*) |
| Demineralized water q.s.f. | 100 g | 100 g | 100 g | 100 g | 100 g |

(*): dyeing support (2) as defined above

At the time of use, each composition was mixed with an equal weight of hydrogen peroxide at 20 volumes (6 wt. %). A final pH of 9.5 was obtained.

Each mixture obtained was applied to locks of grey hair comprising 90% white hairs. After a period of coloration time of 30 minutes, the locks were rinsed, washed with a standard shampoo, rinsed again, and then dried.

The shades obtained are shown in the following table:

Example

| | 31 | 32 | 33 | 34 | 35 |
|---|---|---|---|---|---|
| Shade | red- | red- | orangey | deep | violet |
| observed | violet | brown | | blue | grey |

What is claimed is:

1. A para-phenylenediamine compound chosen from compounds of formula (I) and the addition salts thereof: 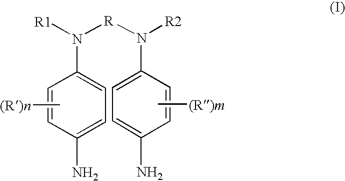
wherein:
R is chosen from branched C₃-C₁₅ alkylene radicals,
R₁ and R₂, which may be identical or different, are chosen from:
hydrogen,
linear and branched C₁-C₆ alkyl radicals, and
linear and branched C₁-C₆ alkyl radicals substituted by at least one radical chosen from hydroxy, C₁-C₄ alkoxy, amino, C₁-C₄ monoalkylamino, and C₁-C₄ dialkylamino radicals,
R′ and R″, which may be identical or different, are chosen from:
C₁-C₆ alkyl radicals,
C₁-C₆ alkoxy radicals,
C₁-C₆ hydroxy-alkoxy radicals,
C₁-C₆ alkoxy(C₁-C₆)alkyl radicals, and
C₁-C₆ mono- or poly-hydroxy alkyl radicals, and
n and m, which may be identical or different, are chosen from integers ranging from 0 to 4.

2. The para-phenylenediamine compound of claim 1, wherein R is chosen from branched C₄-C₁₁ alkylene radicals.

3. The para-phenylenediamine compound of claim 1, wherein R₁ and R₂, which may be identical or different, are chosen from hydrogen and optionally substituted C₁-C₄ alkyl groups.

4. The para-phenylenediamine compound of claim 1, chosen from:

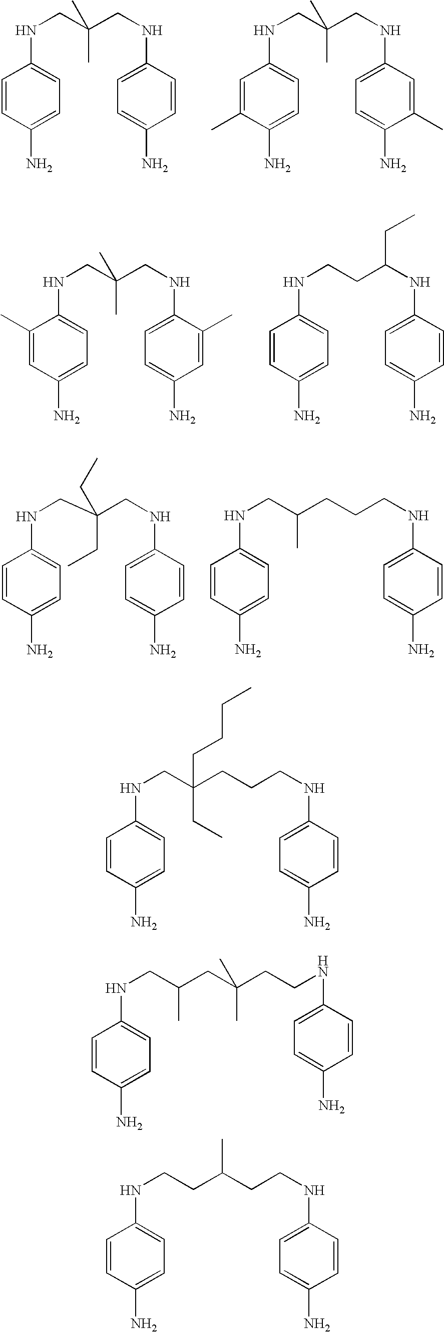

5. The para-phenylenediamine compound of claim 1, wherein the addition salts of the compounds of formula (I) are chosen from hydrochloric acid, hydrobromic acid, sulphuric acid, citric acid, succinic acid, tartaric acid, lactic acid, para-toluene-sulphonic acid, benzene-sulphonic acid, phosphoric acid, acetic acid addition salts, these compounds optionally being in the form of solvates.

6. A dyeing composition comprising, in a medium suitable for dyeing, at least one oxidation base chosen from compounds of formula (I) and the addition salts thereof: 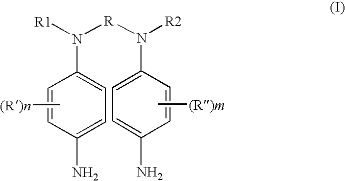
wherein:
R is chosen from branched C₃-C₁₅ alkylene radicals,
R₁ and R₂, which may be identical or different, are chosen from:
hydrogen,
linear and branched C₁-C₆ alkyl radicals, and
linear and branched C₁-C₆ alkyl radicals substituted by at least one radical chosen from hydroxy, C₁-C₄ alkoxy, amino, C₁-C₄ monoalkylamino, and C₁-C₄ dialkylamino radicals,
R′ and R″, which may be identical or different, are chosen from:
C₁-C₆ alkyl radicals,
C₁-C₆ alkoxy radicals,
C₁-C₆ hydroxy-alkoxy radicals,
C₁-C₆ alkoxy(C₁-C₆)alkyl radicals, and
C₁-C₆ mono- or poly-hydroxy alkyl radicals, and
n and m, which may be identical or different, are chosen from integers ranging from 0 to 4.

7. The composition of claim 6, wherein the at least one oxidation base is present in the composition in an amount ranging from 0.0001 wt. % to 20 wt. % relative to the total weight of the composition.

8. The composition of claim 7, wherein the at least one oxidation base is present in the composition in an amount ranging from 0.01 wt. % to 10 wt. % relative to the total weight of the composition.

9. The composition of claim 6, further comprising at least one oxidation coupler.

10. The composition of claim 9, wherein the at least one oxidation coupler is chosen from meta-phenylenediamines, meta-aminophenols, meta-diphenols, naphthalenic couplers, heterocyclic couplers, and the addition salts thereof.

11. The composition of claim 6, further comprising at least one additional oxidation base different from the at least one oxidation base of formula (I).

12. The composition of claim 10, wherein the at least one additional oxidation base is chosen from para-phenylenediamines, bis-phenylalkylenediamines, para-aminophenols, ortho-aminophenols, heterocyclic bases, and the addition salts thereof.

13. The composition of claim 6, further comprising at least one direct dye.

14. The composition of claim 6, wherein the medium suitable for dyeing is chosen from water and mixtures of water and at least one organic solvent.

15. The composition of claim 14, wherein the at least one organic solvent is chosen from linear and branched C₁-C₄ lower alcohols, and aromatic alcohols.

16. The composition of claim 6, further comprising at least one cosmetic additive chosen from antioxidants, penetrants, sequestering agents, perfumes, buffers, dispersants, surfactants, conditioners, film-forming agents, polymers, ceramides, preservatives, lustre agents, opacifiers, vitamins, and provitamins.

17. The composition of claim 16, wherein the at least one cosmetic additive is present in the composition in an amount, for each additive, ranging from 0.01 wt. % to 20 wt. % relative to the total weight of the composition.

18. The composition of claim 6, further comprising at least one oxidizing agent.

19. A method for dyeing keratin fibers, comprising applying at least one dyeing composition to the keratin fibers, for a sufficient time for development of a desired coloration, in the presence of at least one oxidizing agent,
wherein the at least one dyeing composition comprises, in a medium suitable for dyeing, at least one oxidation base chosen from compounds of formula (I) and the addition salts thereof: 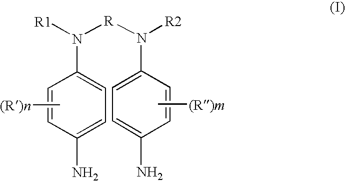
wherein:
R is chosen from branched C₃-C₁₅ alkylene radicals,
R₁ and R₂, which may be identical or different, are chosen from:
hydrogen,
linear and branched C₁-C₆ alkyl radicals, and
linear and branched C₁-C₆ alkyl radicals substituted by at least one radical chosen from hydroxy, C₁-C₄ alkoxy, amino, C₁-C₄ monoalkylamino, and C₁-C₄ dialkylamino radicals,
R′ and R″, which may be identical or different, are chosen from:
C₁-C₆ alkyl radicals,
C₁-C₆ alkoxy radicals,
C₁-C₆ hydroxy-alkoxy radicals,
C₁-C₆ alkoxy(C₁-C₆)alkyl radicals, and
C₁-C₆ mono- or poly-hydroxy alkyl radicals, and
n and m, which may be identical or different, are chosen from integers ranging from 0 to 4.

20. A multi-compartment kit comprising at least one first compartment containing at least one dyeing composition for the dyeing of keratin fibers, and at least one second compartment containing at least one oxidizing agent,
wherein the at least one dyeing composition comprises, in a medium suitable for dyeing, at least one oxidation base chosen from compounds of formula (I) and the addition salts thereof: 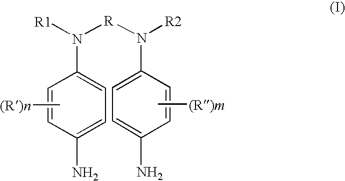
wherein:
R is chosen from branched C₃-C₁₅ alkylene radicals,
R₁ and R₂, which may be identical or different, are chosen from:
hydrogen,
linear and branched C₁-C₆ alkyl radicals, and
linear and branched C₁-C₆ alkyl radicals substituted by at least one radical chosen from hydroxy, C₁-C₄ alkoxy, amino, C₁-C₄ monoalkylamino, and C₁-C₄ dialkylamino radicals,
R′ and R″, which may be identical or different, are chosen from:
C₁-C₆ alkyl radicals,
C₁-C₆ alkoxy radicals,
C₁-C₆ hydroxy-alkoxy radicals,
C₁-C₆ alkoxy(C₁-C₆)alkyl radicals, and
C₁-C₆ mono- or poly-hydroxy alkyl radicals, and
n and m, which may be identical or different, are chosen from integers ranging from 0 to 4.

21. A process for synthesizing a para-phenylenediamine compound of formula (I): 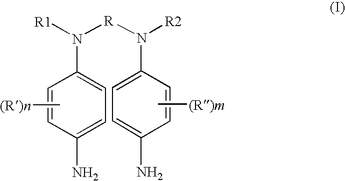
comprising reducing a nitrogen-containing compound of formula (II): 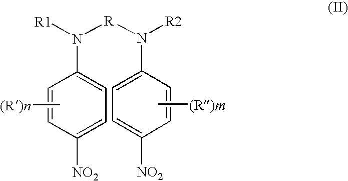
wherein in formulas (I) and (II):
R is chosen from branched C₃-C₁₅ alkylene radicals,
R₁ and R₂, which may be identical or different, are chosen from:
hydrogen,
linear and branched C₁-C₆ alkyl radicals, and
linear and branched C₁-C₆ alkyl radicals substituted by at least one radical chosen from hydroxy, C₁-C₄ alkoxy, amino, C₁-C₄ monoalkylamino, and C₁-C₄ dialkylamino radicals,
R′ and R″, which may be identical or different, are chosen from:
C₁-C₆ alkyl radicals,
C₁-C₆ alkoxy radicals,
C₁-C₆ hydroxy-alkoxy radicals,
C₁-C₆ alkoxy(C₁-C₆)alkyl radicals, and
C₁-C₆ mono- or poly-hydroxy alkyl radicals, and
n and m, which may be identical or different, are chosen from integers ranging from 0 to 4.

22. A nitrogen-containing compound of formula (II): 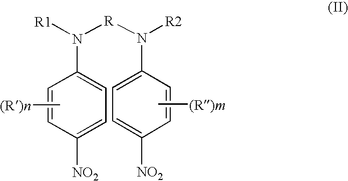
wherein:
R is chosen from branched C₃-C₁₅ alkylene radicals,
R₁ and R₂, which may be identical or different, are chosen from:
hydrogen,
linear and branched C₁-C₆ alkyl radicals, and
linear and branched C₁-C₆ alkyl radicals substituted by at least one radical chosen from hydroxy, C₁-C₄ alkoxy, amino, C₁-C₄ monoalkylamino, and C₁-C₄ dialkylamino radicals,
R′ and R″, which may be identical or different, are chosen from:
C₁-C₆ alkyl radicals,
C₁-C₆ alkoxy radicals,
C₁-C₆ hydroxy-alkoxy radicals,
C₁-C₆ alkoxy(C₁-C₆)alkyl radicals, and
C₁-C₆ mono- or poly-hydroxy alkyl radicals, and
n and m, which may be identical or different, are chosen from integers ranging from 0 to 4;
with the proviso that the compound of formula (II) is not 1,2-propanediamine N,N′-bis(4-nitrophenyl).
